(19) United States
(12) Patent Application Publication
(10) Pub. No.: US 2005/0008663 A1
(43) Pub. Date: Jan. 13, 2005

(54) Topical cream and method of making same

(76) Inventor: Shelah M. Lerma, Plantation, FL (US)

Correspondence Address: Daniel C. Crilly, Esq.;Brinkley, McNerney, Morgan Solomon & Tatum, LLP, New River Center, 200 East Las Olas Blvd., Suite 1900, Fort Lauderdale, FL, 33301, US

(21) Appl. No.: 10/619,371

(22) Filed: Jul. 11, 2003

(51) Int. Cl. A61K 7/06

(52) U.S. Cl. 424/401; 424/74

(57) ABSTRACT

A topical cream includes a water mixture, calendula infused olive oil, cassia oil, geranium oil, and wax. The cream optionally, but preferably, includes grapeseed or olive oil, frankincense oil, myrrh oil, and a violet fragrance. The water mixture preferably includes rosewater and a small amount of glycerin. The cream is made by mixing and heating predetermined amounts of the oils to produce a hot oil mixture. The wax, preferably in grated form, is added to the hot oil mixture to produce a creamy wax mixture. The wax mixture is cooled and blended with the water mixture and fragrance (when used) to produce the cream. The cream provides medicinal, cosmetic, therapeutic, and/or spiritual benefits.

### FIELD OF THE INVENTION

The present invention relates generally to topical skin creams and, in particular, to a hypoallergenic topical skin cream that includes particular essential and other oils to provide beneficial cosmetic and medicinal benefits for the skin.

### BACKGROUND OF THE INVENTION

For decades, people, particularly women, have used lotions and/or creams regularly for medicinal, cosmetic, and/or therapeutic reasons. More recently, people have begun using lotions and creams that contain essential oils. An essential oil is a chemical that can be extracted from a plant and is the source of at least part of that plant's aroma. Essential oils are often used alone or in combination to provide therapeutic healing benefits when applied to the skin and to provide a fragrance that is both appealing and beneficial. Some essential oils are also believed to provide certain spiritual benefits.

The use of essential oils generally in topical creams or lotions is well known. For example, in some shopping malls, there are stores where customers can select essential oils to add to perfumes, colognes, lotions, soaps, and other cosmetic preparations. Since most consumers are not aware of the benefits that essential oils can provide either individually or in particular combinations, the rewards of these self-prepared cosmetics are limited. Further, after creating and purchasing their cosmetic concoctions, customers may later find out that they are allergic to components of the colognes, lotions, or other cosmetic preparations they've created with essential oils, and, therefore, be unable to use them.

Customer-created cosmetics are not the only products with allergy concerns. The ingredients in many commercially available topical creams also cause allergic reactions in their users. For instance, a common allergy to many topical creams is with respect to preservatives that are used in the creams to lengthen their shelf life. Customers' allergies to these preservatives prohibit them from using the creams.

Calendula oil and leaves are known and are used in creams and ointments to provide healing benefits. Calendula oil is commonly produced by combining calendula petals with almond oil in a jar and letting the combination set in a warm ambient environment for an extended period of time (e.g., several weeks). The petals are then removed leaving the oil mixture. One currently available topical ointment containing calendula leaves is described in U.S. Pat. No. 5,843,467 issued to Ambroziewigz. The topical ointment disclosed and described in that patent uses a combination of animal fat, dried plantain leaves, and dried calendula leaves in a specific ratio as an aide for healing skin afflictions and reducing swelling. While this ointment may fulfill its objectives and requirements, its use of animal fat for a base may cause an allergic reaction to sensitive individuals and may anger those who have personal beliefs regarding the use of animal byproducts in commercial products. Further, the ointment fails to include essential oils that provide healing, therapeutic, and/or spiritual benefits.

Therefore, a need exists for a topical cream, and associated method of making same, that includes certain essential oils and provides their attendant therapeutic, healing and spiritual benefits, provides the healing benefits of calendula oil, and can be used by people with skin allergies.

### BRIEF DESCRIPTION OF THE DRAWINGS

FIG. 1 is a flow diagram of steps executed to manufacture a topical cream in accordance with a preferred embodiment of the present invention.

FIG. 2 is a flow diagram of steps executed to manufacture calendula infused olive oil in accordance with a preferred embodiment of the present invention.

### DETAILED DESCRIPTION OF PREFERRED EMBODIMENTS

Generally, the present invention encompasses a topical cream, and an associated manufacturing method, that includes four primary ingredients: a water mixture, an essential oil mixture, calendula infused olive oil, and wax. The water mixture preferably includes rosewater and a small amount of glycerin. The essential oil mixture includes at least cassia oil and geranium oil, and preferably further includes frankincense oil and myrrh oil. The cream may optionally also include grapeseed or olive oil, aloe vera gel, vitamin E, co-enzyme Q10, and/or a fragrance. To produce the cream, the water mixture, aloe vera gel (if used) and a fragrance (if used) are combined, preferably in a blender. The oils are separately combined together with vitamin E (if used) and heated, preferably in a double boiler on low heat. Once the oils are hot, wax and co-enzyme Q10 (if used) are added to the oil mixture. After the wax has melted, the wax/oil mixture is removed from the heat and allowed to cool into a cream. The water mixture and its optional ingredients (if used) are blended or otherwise mixed with the cream to produce the final product. The resulting cream has medicinal benefits (through its inclusion of calendula infused olive oil), therapeutic benefits (through its inclusion of essential oils), cosmetic benefits (through its inclusion of calendula infused olive oil), and, to some users, spiritual benefits (through its inclusion of essential oils, in particular, frankincense oil and myrrh oil). In addition, in its purest form without the various optional ingredients mentioned above, the resulting cream is hypoallergenic, thereby providing the foregoing medicinal, therapeutic, and spiritual benefits to all users, regardless of their skin sensitivities.

One of the benefits that the present invention has over currently commercially-available creams is that it can be used by people who have allergies to components of the commercially-available creams. In its purest form, the cream of the present invention has no preservatives and needs none because the particular combination of elements in the cream disclosed herein preserves the cream and permits a long shelf life. Another benefit of the present invention is that it is very simple in composition and is, therefore, easy to manufacture. An ancillary benefit of the present invention is that the cream promotes spiritual health, particularly when implemented to include frankincense oil and/or myrrh oil.

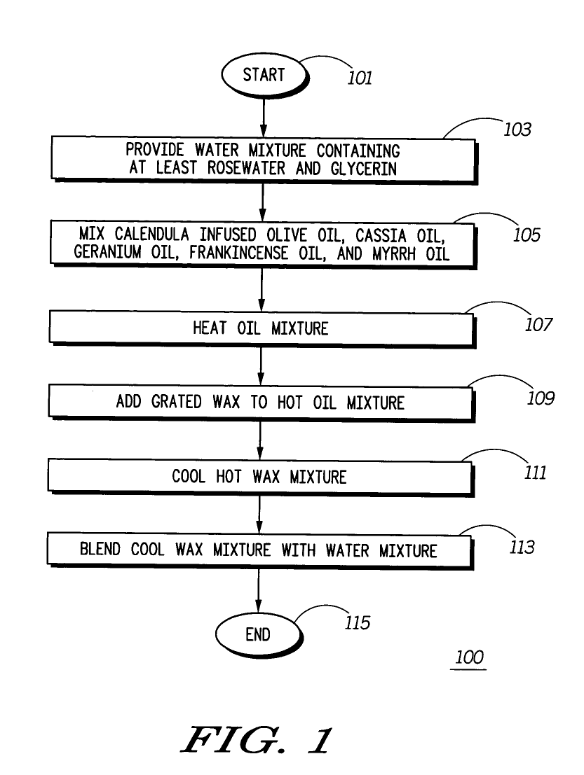

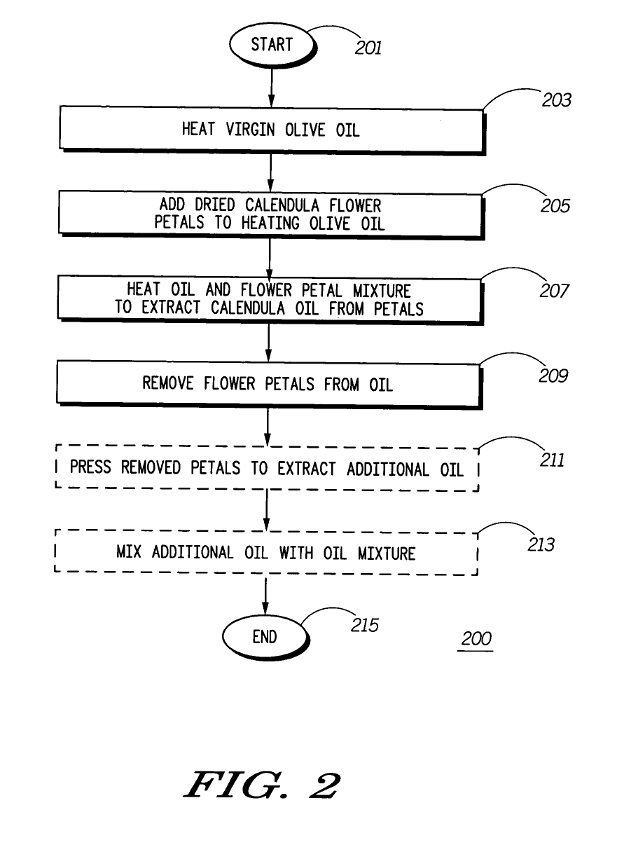

The present invention can be more fully understood with reference to FIGS. 1 and 2. FIG. 1 is a flow diagram 100 of steps executed to manufacture a topical cream in accordance with a preferred embodiment of the present invention. The manufacturing flow begins (101) by providing (103) a water mixture that preferably contains at least rosewater and a small amount of glycerin. In the preferred embodiment, the water mixture occupies at least 32% by volume of the topical cream, with the rosewater preferably accounting for approximately 31% of such volume and the glycerin preferably accounting for the other 1% of such volume. When included, various other non-oil elements are preferably mixed with the water mixture. For example, the cream may optionally include aloe vera gel and a fragrance (e.g., violet). Such ingredients, when included, are preferably mixed with the water mixture. While not required to make the cream, aloe vera gel and/or a fragrance may be included in the cream to improve the scent of the cream and provide moisturizing, healing and other benefits. However, since some people are allergic to one or more of the foregoing optional ingredients, adding such ingredients may reduce the hypoallergenic nature of the cream. In one optional embodiment of the cream, approximately 14% by volume aloe vera gel and approximately 1% or less by volume violet fragrance is mixed with the water mixture for use in manufacturing the cream. All volume percentage references contained herein are with respect to a particular volume of the fully manufactured topical cream.

In addition to providing the water mixture and any optional non-oil elements, the manufacturing flow preferably includes mixing (105) calendula infused olive oil together with a set of essential oils, namely cassia oil, geranium oil and optionally frankincense oil and myrrh oil. Additional oils, such as grapeseed oil and/or olive oil, may be optionally included to fabricate the cream, although the use of grapeseed oil may reduce the hypoallergenic benefits of the cream. The calendula infused olive oil absorbs easily into the skin, healing the skin and preventing excessive dryness. The mixture of essential oils adds to the healing abilities of the cream and also promotes spiritual health. In a preferred embodiment, the oil mixture includes approximately 14% by volume calendula infused olive oil, approximately 33% by volume olive oil and/or grapeseed oil, approximately 0.9% by volume cassia oil, approximately 0.76% by volume geranium oil, approximately 0.14% by volume frankincense oil, and approximately 0.19% by volume myrrh oil.

When included, certain non-oil elements are preferably mixed with the oil mixture prior to heating. For example, the cream may optionally include vitamin E. Such an ingredient, when included, is preferably mixed together with the aforementioned oils. While not required to make the cream, vitamin E may be included in the cream to provide moisturizing and other benefits. However, since some people are allergic to vitamin E, adding such an ingredient may reduce the hypoallergenic nature of the cream. In one optional embodiment of the cream, approximately 1% by volume vitamin E (e.g., 1000 international units per 32 ounces of cream) is mixed with the oils for use in manufacturing the cream.

After the oils and optional vitamin E have been mixed, the oil mixture is heated (107), preferably on a low heat in a double boiler, to a temperature below the boiling point of the mixture. Once the oil mixture is hot, grated wax is preferably added (109) slowly to the hot oil mixture to provide the texture of the cream. The wax also adheres to the skin to protect the skin and prevent evaporation or removal of the other components of the cream. The wax preferably comprises approximately 3% by volume beeswax. For a thicker cream, additional wax may be used (e.g., 5-10% by volume). Analogously, for a thinner cream, less wax may be used (e.g., 2% by volume). In the event co-enzyme Q10 is used as an ingredient in the cream, such ingredient is preferably added to the oil mixture while the oil mixture is hot.

After the wax has been added to the hot oil mixture and has melted therein, the hot wax and oil mixture is cooled (111) to room temperature. The wax mixture is then blended (113) or otherwise mixed with the water mixture, the fragrance (when used) and the aloe vera (when used) to produce the cream, thereby ending (115) the manufacturing flow for purposes of the present invention.

One of ordinary skill in the art will readily recognize that, while the logic flow diagram 100 of FIG. 1 depicts the mixing and heating of the various oils and the creation of the wax mixture occurring logically after the water mixture is provided, the oils may be alternatively mixed and heated, and the wax mixture created, before or contemporaneous with creation of the water mixture. Therefore, the order of blocks 103 and 105-111 of FIG. 1 should not be interpreted to limit the appended claims in any way.

All of the components of the preferred cream are commercially available from various retailers and distributors, with the possible exception of the calendula infused olive oil. Accordingly, FIG. 2 is a flow diagram 200 of steps executed to manufacture the calendula infused olive oil in accordance with a preferred embodiment of the present invention. The manufacturing flow for the calendula infused olive oil begins (201) by heating (203) virgin olive oil on a low heat until the oil is hot, but preferably not boiling, and adding (205) dried calendula (otherwise known as “marigold”) flower petals (e.g., 6 cups of petals for 96 ounces of oil) to the hot olive oil. The oil and flower petals are then heated (207) on a low heat to extract the calendula oil from the flower petals. The flower and oil mixture is preferably heated until the petals turn a golden brown and/or flower petal particles adhere to the oil. In one embodiment containing approximately 96 ounces of olive oil and 6 cups of flower petals, the oil and petals may be heated approximately six hours in a double boiler to extract the calendula oil from the flower petals.

After the calendula oil has been extracted from the flower petals through the aforementioned heating process, the flower petals are removed (209) from the oil mixture to produce the calendula infused olive oil. If additional calendula oil is desired for any reason (e.g., to produce a deeper brown color), the removed petals may be optionally pressed (211) to extract the additional oil and the additional oil may be mixed (213) with the oil mixture to produce the calendula infused olive oil. Once the calendula infused olive oil has been produced, the manufacturing flow ends (215) for purposes of the present invention.

The present invention encompasses a topical cream, and an associated manufacturing method, that includes, in combination, a water mixture, an essential oil mixture, calendula infused olive oil, and wax. With this invention, a moisturizing cream can be produced that provides medicinal, therapeutic and cosmetic benefits though, inter alia, its unique inclusion of calendula infused olive oil. In addition, when the essential oil mixture includes frankincense oil and/or myrrh oil, the cream provides to some users spiritual benefits not found in other creams. Further, when manufactured in its purest form, without optional ingredients such as aloe vera gel, grapeseed oil, co-enzyme Q10, vitamin E and any other potential allergens, the cream of the present invention provides the aforementioned medicinal, therapeutic, cosmetic and/or spiritual benefits in a hypoallergenic form, usable by virtually all individuals.

In the foregoing specification, the present invention has been described with reference to specific embodiments. However, one of ordinary skill in the art will appreciate that various modifications and changes may be made without departing from the spirit and scope of the present invention as set forth in the appended claims. For example, the by-volume percentages of the various ingredients of the cream may be varied by the manufacturer or user to create a desired consistency, fragrance, or other effect. Accordingly, the specification and drawings are to be regarded in an illustrative rather than a restrictive sense, and all such modifications are intended to be included within the scope of the present invention.

Benefits, other advantages, and solutions to problems have been described above with regard to specific embodiments of the present invention. However, the benefits, advantages, solutions to problems, and any element(s) that may cause or result in such benefits, advantages, or solutions to become more pronounced are not to be construed as a critical, required, or essential feature or element of any or all the claims. As used herein and in the appended claims, the terms “comprises,” “comprising” or any other variation thereof is intended to refer to a non-exclusive inclusion, such that a process, method, apparatus, or article of manufacture that comprises a list of elements does not include only those elements in the list, but may include other elements not expressly listed or inherent to such process, method, apparatus, or article of manufacture. All terms used in the appended claims that are not otherwise specifically defined herein should be accorded their ordinary meanings.

1. A topical cream comprising:
a water mixture;
calendula infused olive oil;
cassia oil;
geranium oil; and
wax.

2. The topical cream of claim 1, wherein the water mixture comprises rosewater and glycerin.

3. The topical cream of claim 1, further comprising grapeseed oil.

4. The topical cream of claim 1, further comprising frankincense oil.

5. The topical cream of claim 1, further comprising myrrh oil.

6. The topical cream of claim 1, wherein the wax comprises beeswax.

7. The topical cream of claim 1, further comprising aloe vera gel.

8. The topical cream of claim 1, further comprising vitamin E.

9. The topical cream of claim 1, further comprising co-enzyme Q10.

10. The topical cream of claim 1, further comprising a violet fragrance.

11. A topical cream comprising:
at least 32% by volume water mixture;
approximately 14% by volume calendula infused olive oil;
approximately 0.90% by volume cassia oil;
approximately 0.76% by volume geranium oil; and
a range of approximately 2% to 10% by volume wax.

12. The topical cream of claim 11, wherein the water mixture is approximately 31% by volume rosewater and approximately 1% by volume glycerin.

13. The topical cream of claim 11, further comprising approximately 33% by volume grapeseed oil.

14. The topical cream of claim 11, further comprising approximately 0.14% by volume frankincense oil.

15. The topical cream of claim 11, further comprising approximately 0.19% by volume myrrh oil.

16. The topical cream of claim 11, further comprising approximately 14% by volume aloe vera gel.

17. The topical cream of claim 11, further comprising approximately 1% or less by volume violet fragrance.

18. The topical cream of claim 11, further comprising approximately 33% by volume olive oil.

19. A method of making a topical cream comprising:
providing a water mixture containing at least rosewater and glycerin;
mixing calendula infused olive oil, cassia oil and geranium oil to produce an oil mixture;
heating the oil mixture to produce a hot oil mixture;
adding grated wax to the hot oil mixture to produce a hot wax mixture;
cooling the hot wax mixture to produce a cool wax mixture; and
blending the cool wax mixture with the water mixture.

20. The method of claim 19, further comprising:
mixing at least one of frankincense oil and myrrh oil with the calendula infused olive oil, the cassia oil and the geranium oil to produce the oil mixture.

21. The method of claim 19, further comprising:
heating virgin olive oil;
adding calendula flower petals to the heating virgin olive oil to produce an oil and flower mixture;
heating the oil and flower mixture to extract calendula oil from the calendula flower petals; and
removing the calendula flower petals from the oil and flower mixture after the calendula oil has been extracted to produce the calendula infused olive oil.

22. The method of claim 21, further comprising:
pressing the removed calendula flower petals to extract additional oil; and
mixing the additional oil with the oil and flower mixture to produce the calendula infused olive oil.
